(19) United States
(12) Patent Application Publication
(10) Pub. No.: US 2024/0167720 A1
(43) Pub. Date: May 23, 2024

(54) METHODS, SYSTEMS AND DATA STRUCTURES OF A PLUG-IN VENTILATION CONTROLLER AND UNIVERSAL VENTILATION COVER

(71) Applicant: Futura Technologies, LLC, Pleasant Grove, UT (US)

(72) Inventor: Michael Luebke, Pleasant Grove, UT (US)

(21) Appl. No.: 18/425,966

(22) Filed: Jan. 29, 2024

Related U.S. Application Data
(63) Continuation-in-part of application No. 17/644,334, filed on Dec. 14, 2021, now Pat. No. 11,920,813.
(60) Provisional application No. 63/125,159, filed on Dec. 14, 2020.
(60) Provisional application No. 63/481,848, filed on Jan. 27, 2023.

(51) Int. Cl. F24F 11/88; A61L 2/10; A61L 2/24; A61L 9/015; E03D 9/04; F24F 7/06; F24F 13/20; F24F 110/10; F24F 110/20; F24F 120/14

(52) U.S. Cl. CPC F24F 11/88; A61L 2/10; A61L 2/24; A61L 9/015; E03D 9/04; F24F 7/06; F24F 13/20; A61L 2202/11; A61L 2202/14; A61L 2202/25; A61L 2209/111; A61L 2209/15; A61L 2209/16; F24F 2110/10; F24F 2110/20; F24F 2120/14; F24F 2221/02

(57) ABSTRACT

The present invention relates to a plug-in ventilation control device designed for attachment within a ventilation housing or contained within a cover for the ventilation opening that attaches to the surface around the ventilation opening with this control device providing a platform for additional peripherals such as speakers, lighting, room sanitation systems, aroma diffusion, smart home systems, and voice assistants.

### BACKGROUND

The present disclosure relates to a plug-in exhaust fan controller and a universal cover for exhaust and ventilation systems.

Existing exhaust fan controllers are either built into the device itself or require replacement of the switch controlling the fan. No simple method of upgrading existing fans without wiring exists. Replacement exhaust fan covers exist that cover several models of exhaust fans. There does not presently exist on the market a universal cover compatible with the majority of exhaust fans. In addition, there exists no simple method that transcends brands and models for updating and introducing new features to previously installed exhaust fans. The majority of solutions providing features such as Bluetooth speakers, automatic humidity control, nightlights, additional room lighting, etc. require replacement of the entire exhaust fan system.

### SUMMARY

According to one innovative aspect of the subject matter in this disclosure, a plug-in exhaust fan humidity controller is described. In an example implementation, one general aspect includes a plug-in humidity controller. The plug-in humidity controlling device also includes one or more sensors configured to collect humidity data and temperature data; and attachable housing that connects between a power supply and a venting component, the attachable housing including a command application configured to receive the humidity data and temperature data and cause the venting component to receive power from the power supply responsive to the humidity data and the temperature data.

One general aspect of the ventilation control device is the ability to receive power from the house mains power supply within the ventilation housing and provide power to the ventilation device in response to the environment and power supply as detected by various sensors contained within the device.

Implementations may include one or more of the following features. A humidity controller device where the command application is further configured to cause the venting component to receive power from the power supply responsive to the humidity data exceeding a threshold value. Command application is further configured to cause the venting component to receive power from the power supply responsive to the humidity data increasing beyond a threshold value over a short period of time. The command application is further configured to analyze the humidity data and the temperature data sampled over a period of time, compare the analyzed humidity data and temperature data to a table of events to identify a matching event, and cause the venting component to receive power from the power supply responsive to the matching event being a shower event. The shower event corresponds to data showing an increase in temperature and an increase in humidity over a short period of time. The command application is further configured to analyze the humidity data and the temperature data sampled over a period of time, compare the analyzed humidity data and temperature data to a table of events to identify a matching event, and cause the venting component to stop receiving power from the power supply responsive to the matching event being an air conditioning event. The air conditioning event corresponds to data showing a decrease in temperature and an increase in humidity over a short period of time.

One general aspect includes a power controller. The power controller also includes a power command application that receives a command to control a power supply to an exhaust fan; and a power controller device that causes the power supply to be connected to the exhaust fan in response to the power command application receiving the command to turn on the exhaust fan in response to a criteria being satisfied. Other embodiments of this aspect include corresponding computer systems, apparatus, and computer programs recorded on one or more computer storage devices, each configured to perform the actions of the methods.

Implementations may also include one or more of the following features. A system designed to be triggered by the flushing of a toilet. This system may use sound analytics and a microphone to determine when a flush has occurred. It could also use a water level sensor placed inside the tank of the toilet to determine when a flush has occurred. The water level sensor could be battery operated. It could also be powered using radio waves emitted from the control system. It could relay given states back to the control system using various methods of radio transmission. This system, upon being triggered, could be used to power the ventilation system for a period of time after a trigger event occurs.

Implementations may include one or more of the following features. The power controller where the power controller device is connected between the power supply and the exhaust fan. The power controller device is connected to additional peripheral hardware. The additional peripheral hardware includes one or more of a music playback system, a speaker, a microphone, air freshener system, essential oil delivery system, night light, decorative lights, multicolored light or lights, dehumidification systems, heaters, air conditioners, heat lamps, active noise canceling systems, disinfection systems, and smart home assistant systems, such as a voice assistant. The power controller device is connected to one or more sensors. The one or more sensors include one or more of a humidity sensor, a temperature sensor, a water sensor, a heat sensor, an ambient light sensor, a motion sensor, and a noise sensor, an occupancy sensor, an odor sensor, a smoke sensor, a particulate sensor, a toilet flush sensor. The criteria being satisfied includes the power controller device receiving sensor data from the one or more sensors and determining if the criteria is satisfied if the sensor data exceeds a threshold value. The sensor data that exceeds a threshold value includes humidity data exceeding a humidity value. Implementations of the described techniques may include hardware, a method or process, or computer software on a computer-accessible medium.

Implementations may include one or more of the following features. Micro controllers, computers, speakers, Bluetooth technology, microphones, air fresheners, lights, night lights, colored lights, sound damping materials, dehumidifiers, heaters, air conditioners, heat lamps, Wi-Fi, smart home connectivity, smart assistant features, such as a voice assistant, active noise cancelation, music streaming, humidity sensors, occupancy sensors, motion sensors, odor sensors, temperature sensors, light sensors, particulate sensors, smoke sensors, etc.

The method of powering the device could be accomplished by intercepting the power supplied to the exhaust fan from the home mains power supply. The device could provide automated control of the exhaust fan also includes receiving at a humidity control device, humidity sensor data from a humidity sensor receiving at the humidity control device, temperature sensor data from a temperature sensor; comparing, using a processor the humidity control device, the humidity sensor data and the temperature sensor data to a table of events; determining, using a processor the humidity control device, a match between the humidity sensor data and the temperature sensor data with an event from the table of events; and responsive to determining the match with the event, causing the humidity control device to provide power to a venting component that the humidity control device is connected to. Other embodiments of this aspect include corresponding computer systems, apparatus, and computer programs recorded on one or more computer storage devices, each configured to perform the actions of the methods.

Implementations may include one or more of the following features. The method where the event is a shower event, and the shower event matches with the temperature sensor data increasing and the humidity sensor data increasing. Implementations of the described techniques may include hardware, a method or process, or computer software on a computer-accessible medium.

According to another innovative aspect of the subject matter in this disclosure, a universal exhaust fan cover is described. In an example implementation, one general aspect includes a device which is attached to the structural material around the opening of an exhaust fan. Attachment to the ceiling instead of the fan allows the device to be used universally across all fan brands and models. This may be accomplished by using pins or nails inserted through the device into the substrate or structure around the exhaust fan. This could be but is not limited to wood, drywall, particle board, etc. residing on a ceiling or wall. The pins are oriented at an angle away from or towards each other to create an opposing force to prevent their extraction by a force normal to the mounting surface. This method could be applied at points around the exhaust fan to provide support for the cover as needed. Other methods of attachment to the ceiling for the cover such as but not limited to adhesive tape may also be used. This universal exhaust fan cover may also be composed of a base and cover attached together using magnets or other forms of attachment. The base could be made of various structural materials such as but not limited to metal, plastic, ceramic, etc. The cover could be made of various materials such as but not limited to glass, plexiglass, plastic, etc. The cover could be designed such that various wall and ceiling finishes could be applied to it such as textures and colors via but not limited to plaster, drywall mud, and latex or oil based paints.

Other implementations of one or more of these aspects and other aspects described in this document include corresponding systems, apparatus, and computer programs, configured to perform the actions of the methods, encoded on computer storage devices. The above and other implementations are advantageous in a number of respects as articulated through this document. Moreover, it should be understood that the language used in the present disclosure has been principally selected for readability and instructional purposes, and not to limit the scope of the subject matter disclosed herein.

The disclosure is illustrated by way of example, and not by way of limitation in the figures of the accompanying drawings in which like reference numerals are used to refer to similar elements.

### BRIEF DESCRIPTION OF THE DRAWINGS

FIG. 1 is an example of a plug-in humidity controller system.

FIG. 2 is a block diagram illustrating an example computer system for detecting humidity and powering on a venting component.

FIG. 3 is a block diagram illustrating an example device.

FIG. 4 is an example circuit for controlling a venting component.

FIG. 5 is an example of an exploded view of a universal exhaust fan cover and its corresponding parts.

FIG. 6 is an example of a universal exhaust fan cover with electronic features included.

FIG. 7 is a diagram of components and forces for holding a universal exhaust fan cover to the ceiling.

FIG. 8 is a flowchart for operating a venting component.

FIG. 9 is an example of a system for triggering a venting component when a toilet is flushed.

### DETAILED DESCRIPTION

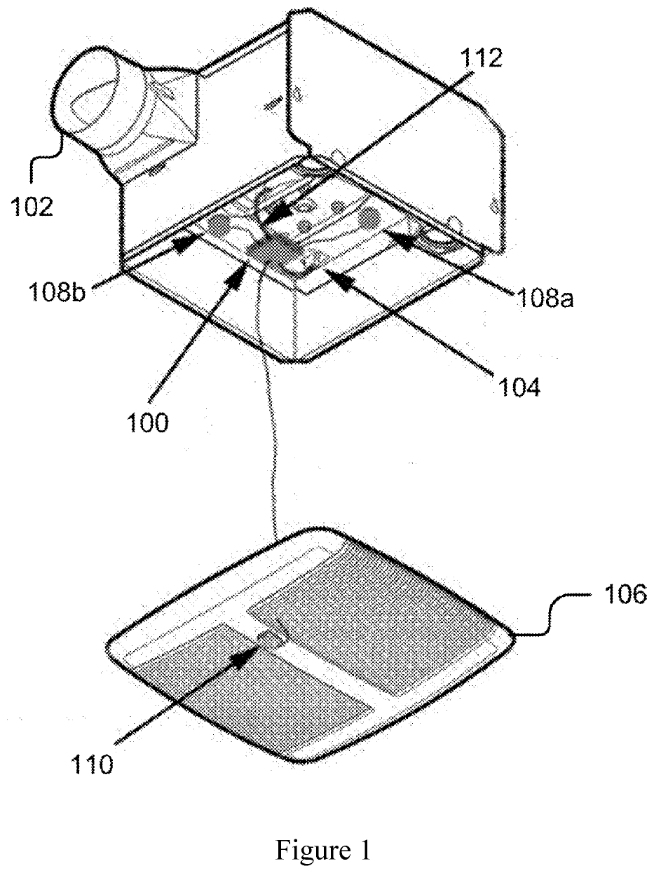

FIG. 1 is an example configuration showing a low-cost solution incorporating a device 100, such as a plug-in humidity controller device, that can be installed in a venting component 102, for example, a bathroom fan such that the device 100 plugs in between the power supply 104 (such as a houses main power supply source) and the power to venting component 112 (such as a power receptacle that previously connected to a bathroom fan) and controls the venting component 102 based on various readings received from sensors connected to the device 100, such as humidity and temperature. In some implementations, the device 100 can also control other peripheral devices, such as a light 110, speaker 108, and or smart home connectivity such as amazon Alexa control or google home control as described elsewhere herein. As shown in FIG. 1, the configuration may also include a cover 106 that can be positioned onto the venting component 102 to cover the internals of the fan 102 and/or the device 100. In some implementations, the cover 106 may be connected to the device 100 and include one or more peripheral devices, such as a light 110 that may be controlled by the device 100.

The device 100 may be a plug-in humidity exhaust controller that is configured to interrupt the regular power from an AC voltage source in a conventional venting component 112, such as a bathroom fan and control the conventional component 112 based on various readings from the device 100. The device 100 may be configured for a simple installation that can be performed without hiring expensive professionals for the installations and can generally be installed in most common venting components 102 simply by connecting the AC voltage source (power supply 104) to the device 100 instead of the venting component 102 and then connecting the power to the venting component 112 to the device. When the device 100 determines that the venting component 112 (e.g., a motor of the venting component 102), then the device 100 provides power to the venting component 112. In some implementations, the device 100 may determine that the venting component 102 should be turned on in response to a sensor reading (such as a temperature or humidity reading from a sensor 210 that exceeds a specific threshold reading). In further implementations, the device 100 may include a humidity application 214 that can perform additional analytics, such as determining when an air conditioner is running rather than a shower and use those analytics in addition to the sensor 210 readings in order to operate the venting component 102.

In some implementations, the device 100 may be configured to mount within an interior area of the venting component 102 such that the device 100 is hidden from view (such as behind a cover 106 of the venting component 102). The device 100 may be configured to mount in such a way as to not impede the operation of a motor or other device within the venting component 102.

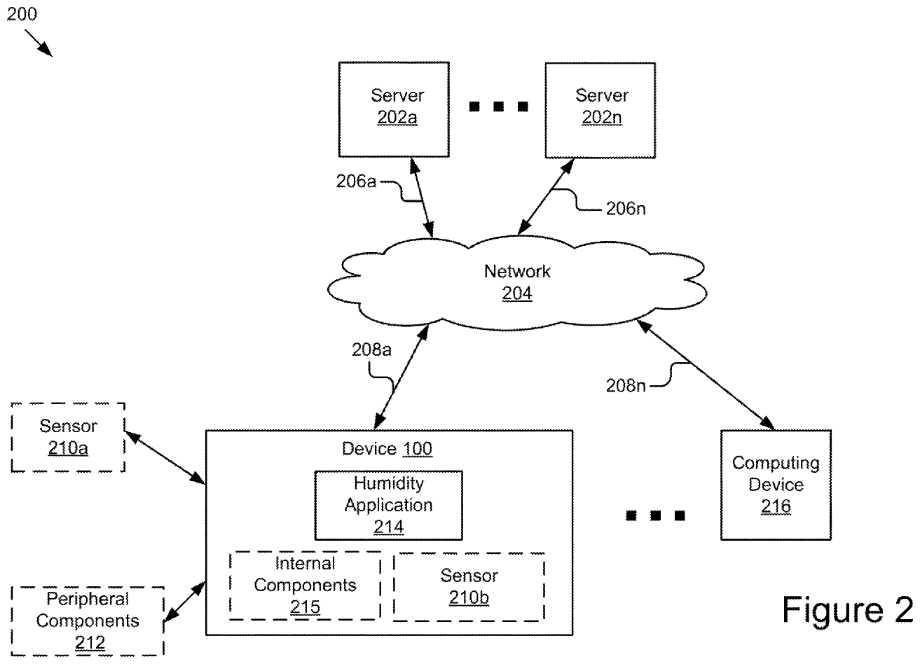

FIG. 2 is a block diagram illustrating an example system 200 for controlling a venting component 102 or other devices with the device 100. The illustrated system 200 includes a device 100, computing device 216, and servers 202a . . . 202n (also referred to individually and collectively as 202), which are communicatively coupled via a network 204 for interaction with one another. For example, the device 100 and/or computing device 216 may be respectively coupled to the network 204 via signal lines 208a . . . 208n. The servers 202a . . . 202n may be coupled to the network 204 via signal lines 206a . . . 206n, respectively. The use of the nomenclature “a” and “n” in the reference numbers indicates that any number of those elements having that nomenclature may be included in the system 200.

The network 204 may include any number of networks and/or network types. For example, the network 204 may include, but is not limited to, one or more local area networks (LANs), wide area networks (WANs) (e.g., the Internet), virtual private networks (VPNs), mobile (cellular) networks, wireless wide area network (WWANs), WiMAX® networks, Bluetooth® communication networks, peer-to-peer networks, other interconnected data paths across which multiple devices may communicate, various combinations thereof, etc.

The computing device 216 may have processing and communication capabilities. For instance, a computing device 216 may include a processor (e.g., virtual, physical, etc.), a memory, a power source, a network interface, and/or other software and/or hardware components, such as front and/or rear facing cameras, display, graphics processor, wireless transceivers, keyboard, camera, speaker, sensors, firmware, operating systems, drivers, various physical connection interfaces (e.g., USB, HDMI, etc.). The computing device 216 may couple to and communicate with one another and the other entities of the system 200 via the network 204 using a wireless and/or wired connection. In some implementations, the computing device 216 may be a connected home device, such as an Amazon Alexa or Google Home smart device and the computing device 216 may be capable of receiving commands from the device 100 via the network 204.

The device 100 may have processing and/or communication capabilities, for instance, a device 100 may include a processor (e.g., virtual, physical, etc.), a memory, a power source, a network interface, and/or other software and/or hardware components, such as display, graphics processor, wireless transceivers, keyboard, camera, sensors, firmware, operating systems, drivers, various physical connection interfaces (e.g., USB, HDMI, etc.). The device 100 may couple to and communicate with one another and the other entities of the system 200 via the network 204 using a wireless and/or wired connection.

As depicted in FIG. 2, the device 100 may include a humidity application 214, and in some implementations a sensor 210b and/or internal components 215. The humidity application 214 can receive readings from various connected sensors 210 and determine based on those readings when to operate a venting component 102.

The servers 202 may each include one or more computing devices having data processing, storing, and communication capabilities. For example, the servers 202 may include one or more hardware servers, server arrays, storage devices and/or systems, etc., and/or may be centralized or distributed/cloud-based. In some implementations, the servers 202 may include one or more virtual servers, which operate in a host server environment and access the physical hardware of the host server including, for example, a processor, memory, storage, network interfaces, etc., via an abstraction layer (e.g., a virtual machine manager).

The servers 202 may include software applications operable by one or more computer processors of the servers 202 to provide various computing functionalities, services, and/or resources, and to send data to and receive data from the device 100 and/or computing device 216. For example, the software applications may provide functionality for connected smart home controls; internet searching; social networking; web-based email; blogging; micro-blogging; photo management; video, music and multimedia hosting, distribution, and sharing; business services; news and media distribution; user account management; or any combination of the foregoing services. It should be understood that the servers 202 are not limited to providing the above-noted services and may include other network-accessible services.

The system 200 in FIG. 2 may also include one or more external sensors 210a, such as external temperature or humidity sensors that can be connected (either wirelessly via the network 204 or via a wired connection to the device 100). The external sensors 210a can detect various sensor readings from a location separate from the device, such as to improve humidity control sensing (e.g., by not having a sensor hidden behind a vent cover 106 and instead having the sensor 210a closer to a shower head, etc.). In further implementations, the external sensors 210a may be temperature sensors that can be placed at various locations throughout a building to change the venting components 102 operation based on the various readings.

The system 200 in FIG. 2 may also include other peripheral components, such as lights, speakers, etc. that can be wirelessly or wired connected to the device 100 and send/receive commands from the device 100 as described elsewhere herein.

It should be understood that the system 200 illustrated in FIG. 2 is provided by way of example, and that a variety of different system environments and configurations are contemplated and are within the scope of the present disclosure. For instance, various functionality may be moved from a server to a client, or vice versa and some implementations may include additional or fewer computing devices, services, and/or networks, and may implement various functionality client or server-side. Further, various entities of the system 200 may be integrated into a single computing device or system or additional computing devices or systems, etc.

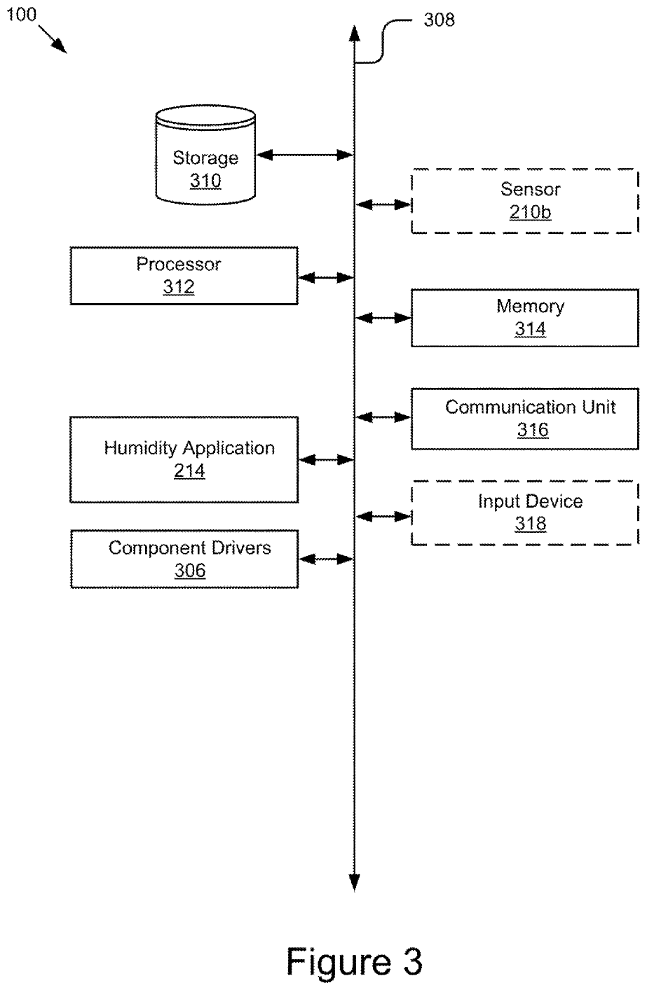

FIG. 3 is a block diagram of an example device 100. As depicted, the device 100 may include a processor 312, memory 314, communication unit 316, component drivers 306, and an input device 318, which are communicatively coupled by a communications bus 308.

The processor 312 may execute software instructions by performing various input/output, logical, and/or mathematical operations. The processor 312 has various computing architectures to process data signals including, for example, a complex instruction set computer (CISC) architecture, a reduced instruction set computer (RISC) architecture, and/or an architecture implementing a combination of instruction sets. The processor 312 may be physical and/or virtual and may include a single core or plurality of processing units and/or cores.

The memory 314 is a non-transitory computer-readable medium that is configured to store and provide access to data to the other elements of the device 100. In some implementations, the memory 314 may store instructions and/or data that may be executed by the processor 312. For example, the memory 314 may store the humidity application 214 and the component drivers 306. The memory 314 is also capable of storing other instructions and data, including, for example, an operating system, hardware drivers, other software applications, data, etc. The memory 314 may be coupled to the bus 308 for communication with the processor 312 and the other elements of the device 100.

The communication unit 316 may include one or more interface devices (I/F) for wired and/or wireless connectivity with the network 204 and/or other devices. In some implementations, the communication unit 316 may include transceivers for sending and receiving wireless signals. For instance, the communication unit 316 may include radio transceivers for communication with the network 206 and for communication with nearby devices using close-proximity (e.g., Bluetooth®, NFC, etc.) connectivity. In some implementations, the communication unit 316 may include ports for wired connectivity with other devices. For example, the communication unit 316 may include a CAT-5 interface, Thunderbolt® interface, FireWire™ interface, USB interface, etc.

The input device 318 may include any device for inputting information into the device 200. In some implementations, the input device 318 may include one or more peripheral devices. For example, the input device 318 may include a keyboard (e.g., a QWERTY keyboard), a pointing device (e.g., a mouse or touchpad), microphone, a camera, etc. In some implementations, the input device 318 may include a touch-screen display capable of receiving input from the one or more fingers of the user. For instance, the functionality of the input device 318 and the display may be integrated, and a user of the computing device 216 may interact with the computing device 216 by contacting a surface of the display using one or more fingers. In this example, the user could interact with an emulated (i.e., virtual or soft) keyboard displayed on the touch-screen display by using fingers to contact the display in the keyboard regions. In further implementations, the input device 318 may be a separate computing device 216 that is connected to the device 200 via the network 204 and can send various input commands via the network 204.

The humidity application 214 includes software and/or logic for identifying one or more triggering events to turn on or off the venting component 102, such as by receiving sensor 210 information and determining triggering events based on the sensor 210 information. The triggering events may be based on environmental data that corresponds to different events occurring, such as the AC being turned on, a toilet being flushed, a shower being started, etc. The activity application(s) 214 may be coupled to the sensor 210 via the processor 312 and/or the bus 308 to receive the information. In some implementations, the humidity application 214 includes a power command application that receives commands to control a power supply for a venting component 102, such as an exhaust fan. In some implementations, the humidity application 214 may check for if there is power available or a power availability at the power supply before turning on the venting component 102. The humidity application 214 can also be referred to herein as the power command application. In some implementations, the humidity application 214 includes a power controller device that causes the power supply to be connected to the venting component 102 in response to the power command application determining various matches of events.

The component drivers 306 includes software storable in the memory 314 and operable by the processor 312 to control/operate various internal components 215 and/or peripheral components 212. For example, the component drivers 306 may be software drivers executable by the processor 312 for signaling the peripheral component 212 representing a light to turn on or off, etc. The component drivers 306 are capable of controlling various features of the internal components 215 and/or external components 212.

The storage 310 is an information source for storing and providing access to stored data, such as a database of historical readings, routines, and/or triggering events, user profile information, community developed virtual routines, virtual enhancements, etc., object data, calibration data, and/or any other information generated, stored, and/or retrieved by the activity application(s) 214.

In some implementations, the storage 310 may be included in the memory 314 or another storage device coupled to the bus 308. In some implementations, the storage 310 may be or included in a distributed data store, such as a cloud-based computing and/or data storage system. In some implementations, the storage 310 may include a database management system (DBMS). For example, the DBMS could be a structured query language (SQL) DBMS. For instance, storage 310 may store data in an object-based data store or multi-dimensional tables comprised of rows and columns, and may manipulate, i.e., insert, query, update, and/or delete, data entries stored in the verification data store using programmatic operations (e.g., SQL queries and statements or a similar database manipulation library). Additional characteristics, structure, acts, and functionality of the storage 310 is discussed elsewhere herein.

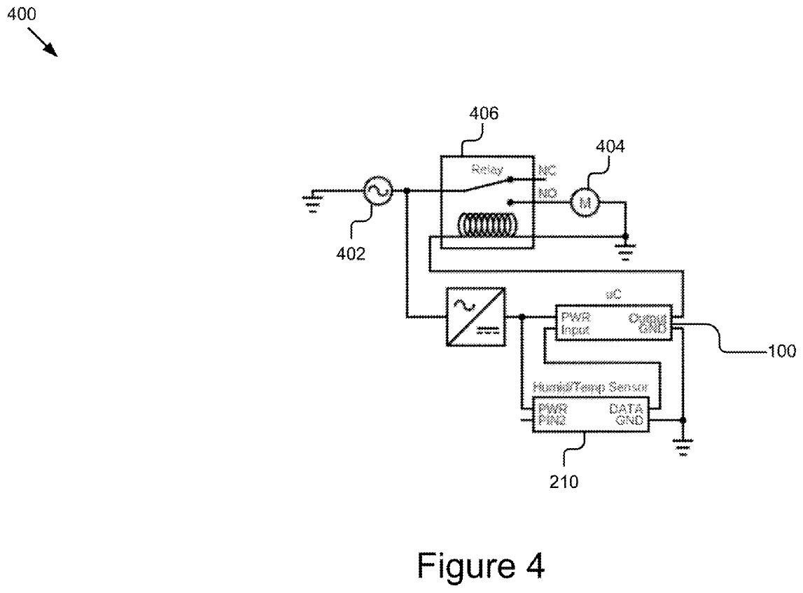

FIG. 4 depicts an example circuit 400 that could be used with the device 100 to control the operation of a venting component. As shown in the example circuit 400, the AC voltage source 402 is connected to the motor 404 of the venting component 102 via a relay 406. The device 100 and the sensor 210 control when that relay 406 provides power to the motor 404 from the AC voltage source 402. In some implementations, this device 100 can be installed in preexisting venting components 102, such as a regular bathroom fan that is already installed in a person's house. The installation merely requires installing the device 100 between the original power supply 104 and the power to the venting component 112. This allows for the device 100 to be easily installed with existing venting components 102. In some implementations, the venting component 102 may be a device other than a bathroom fan, such as a high/low pressure safety van, a vent hood, an oven range vent, etc. In some implementations, the venting component 102 operates by a device other than a motor 404, such as a low pressure-high pressure exchange, vacuum, etc. In those implementations, the power supply to the device operating the exchange or vacuum is used instead of the motor 404 as shown in FIG. 4.

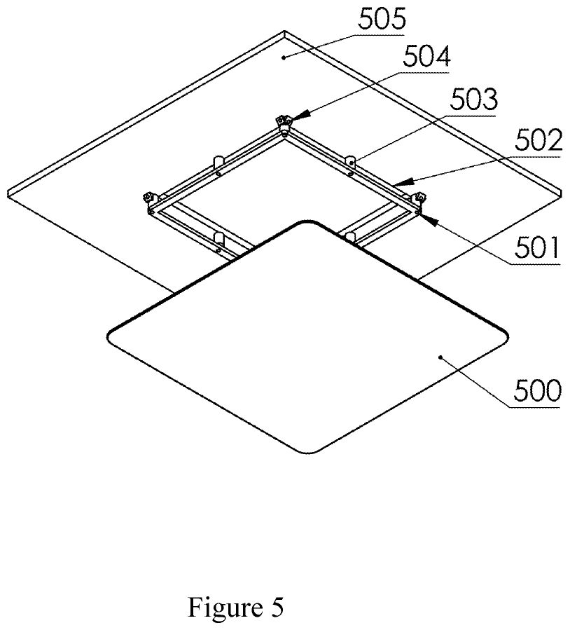

FIG. 5 is an example of a universal exhaust fan cover consisting of components 500, 501, and 503 mounting to ceiling 505 surrounding vent opening 502. The base 503 is attached to ceiling 505 by means of nails, screws, or pins 504 pushed through the base 503 and into the ceiling 505 while the base 503 is held against the ceiling 505, such as to keep the cover 500 stationary. The cover 500 is then attached to the base 503 through magnetic force provided by magnets 501 or other reasonable attachment mechanisms known in the art. In some implementations, magnets 501 may be physically attached to base 503 and cover 500 during manufacturing, such as by attaching into the surface of the cover 500 such as by molding during manufacturing, or adhesives after the cover 500 has been manufactured. The magnets 501 are oriented such that the magnets 501 in the cover 500 are attracted to the magnets 501 in the base 503. The attachment of the magnets 501 to the cover 500 and the base 503 can be accomplished via heat, vibration, over molding or adhesive.

The cover 500 can be composed of various materials or a composite of many materials. Some examples of the materials the cover 500 can be composed of are plastics, glass, metals, and ceramic. The materials composing the cover 500 can go through various processes to change the appearance or finish. One such example could be a thermoplastic cover with a roughened surface prepared to accept various finish treatments to match those applied to the ceiling 505. Some cover 500 finish treatments could include but are not limited to, texturing using drywall mud or plaster, and painting with an oil or water based paint.

The base 503 can be composed of various materials or a composite of many materials. Some examples of materials the base 503 can be composed of include plastics, metals, or ceramics.

The nails or pins 504 can be composed of various materials or a composite of multiple materials. Some example materials the nails or pins 504 can be composed of are various metals, plastics and ceramics. The nails or pins 504 can be sharpened at one end by various methods including stamping, machining, forming etc. The other end of the nails or pins 504 can be blunt and flat or enlarged in diameter by various means.

In various iterations the cover 500 and base 503 can be manufactured as one unit without the need for the magnets 501 to connect them. In such an arrangement the entire unit can be mounted to the ceiling 505 at one time instead of separately mounting the base 503 to the ceiling, and afterwards mounting the cover 500 to the base 503.

In various iterations the cover 500 can house various sensors, electronics, computers, speakers, lights, and control systems. In one embodiment the cover could house an occupancy sensor and/or a UV light, such as a built-in UV light. The system can be designed such that when the bathroom is unoccupied the UV light is initialized for a period of time to sanitize the room. In another embodiment an air freshener can be implemented in the cover and at given intervals is dispersed into the room. In another embodiment, essential oils could be housed within the cover in addition to a dispersal system. A user can give a command through a remote control, a cell phone app, or verbal command, which would trigger the release of essential oils into the air. This system can also be accompanied with multicolored lights to create a desired room ambiance.

In another iteration the device can include microphones, a computer processor and a relay system. The computer processor analyzes input from the microphones using an algorithm. The algorithm is designed to provide a positive identification of the sound of a toilet flushing. Upon detection of a toilet flush, the computer system starts a timer and enables power to the ventilation device. Upon completion of the timer, power to the ventilation device is disabled.

In some embodiments the cover 500 may contain a humidity exhaust controller that is configured to interrupt the regular power from an AC voltage source in a conventional venting component, such as a bathroom fan and control the conventional component based on various readings received by the device 100. The device 100 may be configured for a simple installation that can be performed without hiring expensive professionals for the installations and can generally be installed with most common venting components simply by connecting the AC voltage source (power supply 104) to the device 100 instead of the venting component 102 and then connecting the power to the venting component 112 to the device. When the device 100 determines that the venting component 404 (e.g., a motor of the venting component), then the device 100 provides power to the venting component 112. In some implementations, the device 100 may determine that the venting component 112 should be turned on in response to a sensor reading (such as a temperature or humidity reading from a sensor 210 that exceeds a specific threshold reading). In further implementations, the device 100 may include a humidity application 214 that can perform additional analytics, such as determining when an air conditioner is running rather than a shower and use those analytics in addition to the sensor 210 readings in order to operate the venting component 404.

In some implementations, the cover 500 may include one or more ventilation holes or openings that allow air to pass through the cover 500. In some implementations, the ventilation holes can be designed specifically so the baffle space allows air to flow through with reduced noise. In further implementations, the ventilation holes may be larger to reduce the number of times it needs to be cleaned from dust clogging the ventilation holes.

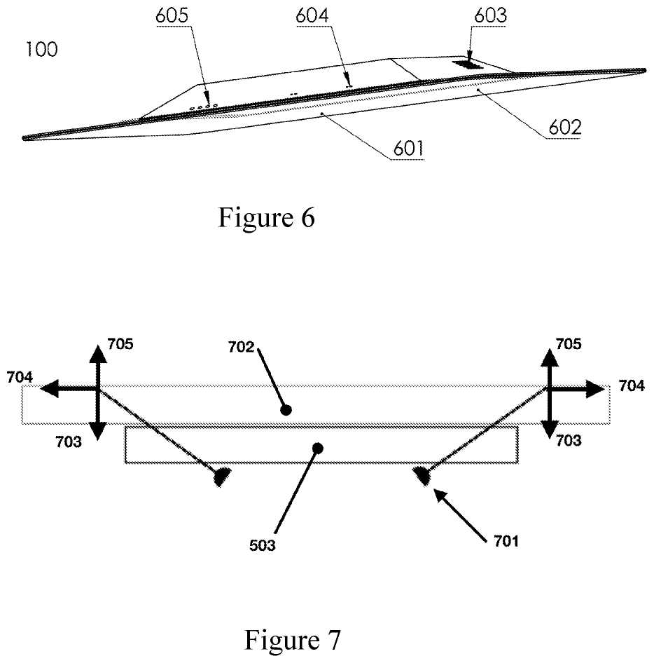

FIG. 6 depicts a cover 500 that may include one or more peripheral components that may incorporate various “smart” features. For example, in one implementation, a lighting source 601 can be incorporated into the cover 500. The lighting source 601 could have power provided from the AC power supply 104 and the device 100 can control when the lighting source 601 is turned on/off. For example, the lighting source 601 could be a night light that is configured to use a separate light sensor 210 (not shown), such as an ambient light sensor, to detect low light conditions and the device 200 causes the lighting source 601 to turn on when the low light condition is detected. In further implementations, the lighting source 601 could be turned on using a motion sensor 210 (not shown) and the device 100 causes the lighting source 601 to turn on in response to the motion being detected. In further implementations, the condition of both motion and low light being detected could trigger the device 100 to cause the lighting source 601 to turn on for a period of time. In some implementations, the peripheral device may be a night light or other smart light. In some implementations the peripheral device may be an aromatic oil, essential oil, or other dispersal system substance.

In some implementations, the lighting source could include both RGB LED indicator lights 602 and/or white illumination lights 601 and the device 100 could determine which lights to turn on based on various sensor and/or alert readings. For example, if the low light and motion is detected, the device 100 could turn on the white illumination lights 601 at a low light setting. In further example, if a separate device, such as a smart home connected device detects a security alert, the device 100 could receive that signal from the smart home connected device and cause a bright illumination to occur on the white illumination lights 601. In further implementations, the device 100 could use the various RGB LED indicator lights 602 to display various patterns or alerts. For example, the device 100 could cause the RGB LED indicator lights 602 to display a small blue light on the RGB LED indicator lights 602 when the venting component 404 is operating. In further implementations, the device 100 could cause the RGB LED indicator light 602 to display a ring of lights that disappear as a timer counts down to automatic shutoff of the venting component 404. In some implementations, the RGB LED indicator lights 602 could act as a built-in night light or built-in colored lighting element.

In some implementations, the device 100 may include a music playback system consisting of one or more speakers incorporated within the cover 500, such as speakers 603. These speakers 603 may receive their audio signal from a separate computing device 216, such as a mobile phone or other device using various transmission technologies such as but not limited to Bluetooth, Apple AirPlay, etc. In some implementations, the device 100 may be designed to reduce the sound of the fan motor 404 or other venting apparatus while the music playback system is operating. In further implementations, the device 100 may sample the existing venting component 404 noise (such as by using a microphone 604) during operation and use noise cancellation algorithms with the sampled venting component 404 noise in order to employ noise reduction when the speakers 603 are being used with the music playback system. The noise cancellation algorithms can create noise patterns to diminish the sound of the fan. In further implementations, in a bathroom setting, the device 100 can detect motion using a motion sensor and turn on the venting apparatus while the bathroom is being used. While the venting apparatus is on, the device 100 can cause music or white noise to play through the speakers 603 in order to both diminish the sound of the venting apparatus, while also creating a comfortable atmosphere in the bathroom. In some implementations, once the motion is no longer detected using the motion sensor, the device 100 can turn off the music/white noise and turn off the venting apparatus after a period of time.

In some implementations, the device could include a built in dispersal system for aromatic oils and/or other aromatic substances. Based on various criteria being met, such as environmental event data, or the venting component being turned on, the built-in dispersal system could dispense the aromatic oils and substances.

In some implementations, the device 100 may also contain one or more microphones 604 to receive audio instructions allowing it to accept voice commands or other audio samples. These commands may be used to control the device 100 itself or other devices connected through a unifying ecosystem, such as a commonly referred to as a smart home. Various services such as Amazon Alexa or Google Home could be integrated with the device 100 to accomplish the smart home ecosystem.

FIG. 7 shows a close up view of an attachment point between the base 503, and the ceiling 702 using a nail or pin 701. The nail or pin 701 is pushed through the base 503 at an angle neither parallel nor perpendicular to the surface of the ceiling 702 and into the ceiling 702 following this same path. The angle of the nail or pin 701 resists gravitational forces normal to the surface 703 of the ceiling 702 by creating counter forces 704 parallel to the surface of the ceiling 702 and 705 perpendicular to the ceiling 702 that counter the gravitational force 703. By placing nails or pins 701 with angles oppositional to each other the parallel forces 704 are canceled out and the object 503 is held in place against the ceiling 702.

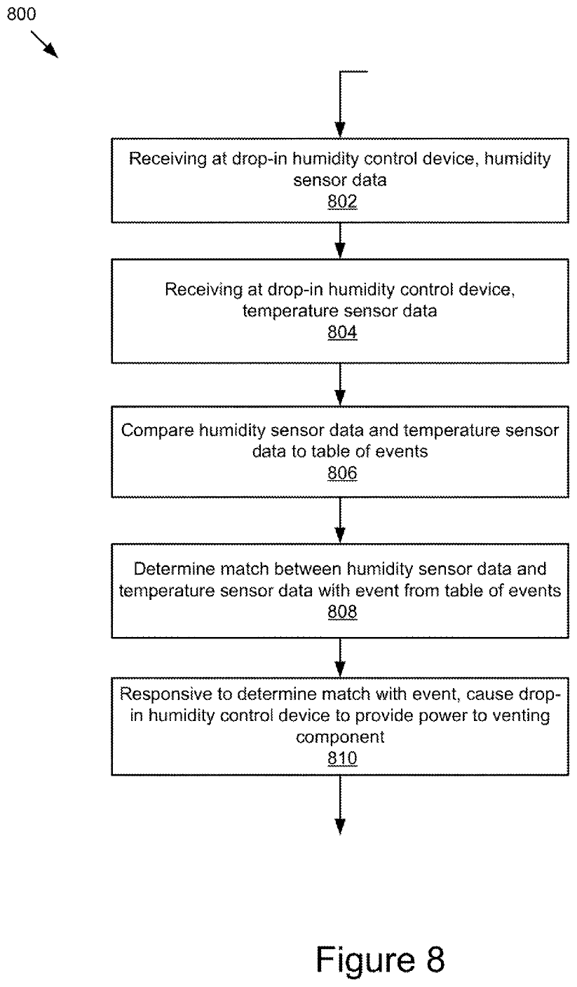

FIG. 8 is a flowchart 800 of an example method for providing power to a venting component. At 802, the humidity application 214 receives humidity sensor data from a humidity sensor 210. In some implementations, the humidity sensor 210 is incorporated into an exterior surface of the cover 500. The humidity sensor 210 captures humidity data to determine when there are increases in humidity, such as when a shower or bath is happening in a bathroom or when an air conditioning unit is operating. The humidity sensor 210 captures this humidity data over a period of time and can detect increased levels of humidity.

At 804, the humidity application 214 receives temperature sensor data from a temperature sensor 210. In some implementations, the temperature sensor 210 can be internal to the device 100 or external, such as remote or mounted on an exterior surface of a cover 500. In some implementations, multiple temperature sensors 210 may be used at various locations, and the humidity application 214 may ignore values from certain locations depending on the data needed or average the values of various temperature sensors 210. In some implementations, temperature sensor 210 captures temperature data that is then analyzed by the humidity application 214 to determine an increase/decrease in temperature or heat, such as when a shower or bath is happening or when an air conditioning unit is operating.

At 806, the humidity application 214 compares the humidity sensor data and the temperature sensor data to a table of events. The table of events includes various events that correspond to different sensor readings. For example, a shower/bath event is more correlated to an increase in both temperature and humidity over a short period of time. In another example, an air conditioning event may correlate to an increase in humidity and corresponding decrease in temperature. Over time, the humidity application 214 can characterize various events at various levels of sensor detail. For example, over time the humidity application 214 can begin to model when a person is in a room based on a small increase in temperature from body heat or what the data for events looks like at different times of day and during different seasons. The model of events can be improved over time as data is sampled and various machine learning algorithms could be used to further interpret events. In some implementations, user input via a computing device 216 can also be used to categorize different events. In some implementations, push notifications can be sent to a computing device to answer questions about what is occurring in order to further characterize data in substantially real-time.

At 808, the humidity application 214 can determine a match between humidity sensor data and temperature sensor data with an event from the table of events. The humidity application 214 can match the provided data to characterized events as described above and additionally identify new or outlier events that don't match any event models. Once an event is matched, the humidity application 214 can determine an appropriate action for the event. In some implementations, the appropriate action is to cause the venting component 404 to turn on a fan for a period of time, such as when a shower is occurring. In further implementations, the appropriate action is to not turn on the venting component 102, such as when an air conditioner is running, causing an increase in humidity. In further implementations, additional sensor data, such as from light sensors, motion sensors, water sensors, can also be used to characterize various events and/or routines the device 100 can cause to be executed and turn on/off various peripheral components 212.

At 810, the humidity application 214 causes the plug-in humidity control device to provide power to the venting component 102 responsive to determining that turning on the power is an appropriate response to the event. In some implementations, this causes power to be provided to the venting component 102 and allows the fan to turn on and vent out the humidity for a period of time. By being able to automatically turn on the fan in substantially real-time as humidity levels are detected, the humidity can be removed without causing long-term damage to the room. Additionally, by being able to determine which events are appropriate to turn on the fan, the device 100 will also not turn on the fan to vent when not required, such as when air conditioning is cooling a room.

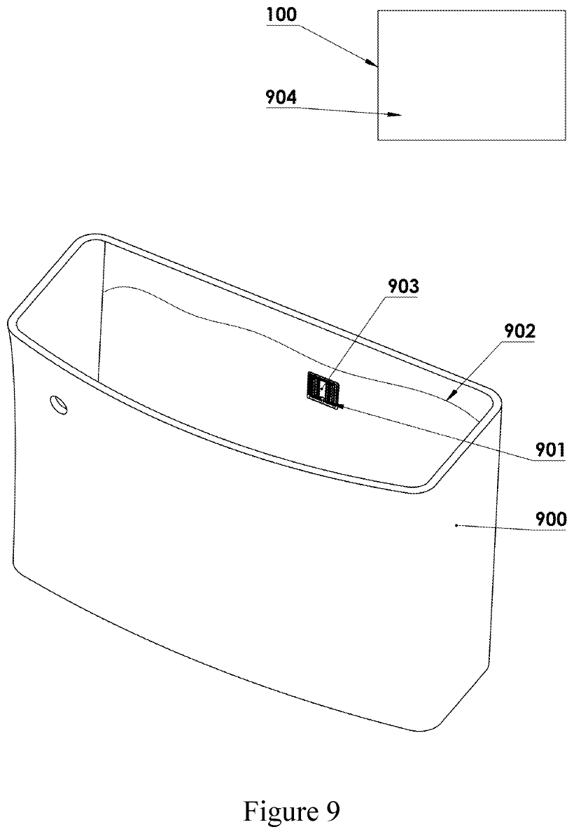

FIG. 9 illustrates another embodiment of the device 100 wherein the exhaust fan can be controlled in response to a toilet flush. The device 100 can include a radio transmission device 904, a processor and a power relay. The radio transmission device 904 emits a consistent radio signal. A radio frequency identification device 903 hereafter referred to as RFID, can be placed inside a toilet tank 900. The RFID device 903 is positioned inside the toilet tank such that when the tank is full of water, the RFID device is fully submerged. The RFID device 903 is designed to collect energy from a radio signal emitted from the radio transmission device 904. When sufficient energy is collected, the RFID 903 is designed to emit a radio signal. The radio signal emitted from the RFID 903 contains a digital encoding identifying the RFID 903. The radio transmission device 904 detects this radio signal and indicates to a processor the device is present. Two contact points 901 connected to the power collection circuit in the RFID 903 can be exposed to the water in the toilet tank. When the water level 902 is full the two contact points 901 will allow current to flow between them and will drain the energy collected by the RFID 903 from the radio transmission device 904 preventing it from broadcasting. When the toilet is flushed the water level 902 drops below the RFID 903 and the contact points 901 prevent current from flowing and allowing the RFID 903 to broadcast its signal. In receiving a signal from the radio transmission device 904 the processor begins an internal timer and enables the relay 406 to power the ventilation device 102. Upon completion of the timer, the processor disables the relay 406 and subsequently the ventilation device 102.

It should be understood that the above-described example activities are provided by way of illustration and not limitation and that numerous additional use cases are contemplated and encompassed by the present disclosure. In the above description, for purposes of explanation, numerous specific details are set forth in order to provide a thorough understanding of the present disclosure. However, it should be understood that the technology described herein may be practiced without these specific details. Further, various systems, devices, and structures are shown in block diagram form in order to avoid obscuring the description. For instance, various implementations are described as having particular hardware, software, and user interfaces. However, the present disclosure applies to any type of computing device that can receive data and commands, and to any peripheral devices providing services.

In some instances, various implementations may be presented herein in terms of algorithms and symbolic representations of operations on data bits within a computer memory. An algorithm is here, and generally, conceived to be a self-consistent set of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout this disclosure, discussions utilizing terms including “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Various implementations described herein may relate to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, including, but is not limited to, any type of disk including floppy disks, optical disks, CD-ROMs, and magnetic disks, read-only memories (ROMs), random access memories (RAMs), EPROMs, EEPROMs, magnetic or optical cards, flash memories including USB keys with non-volatile memory or any type of media suitable for storing electronic instructions, each coupled to a computer system bus.

The technology described herein can take the form of a hardware implementation, a software implementation, or implementations containing both hardware and software elements. For instance, the technology may be implemented in software, which includes but is not limited to firmware, resident software, microcode, etc. Furthermore, the technology can take the form of a computer program product accessible from a computer-usable or computer-readable medium providing program code for use by or in connection with a computer or any instruction execution system. For the purposes of this description, a computer-usable or computer readable medium can be any non-transitory storage apparatus that can contain, store, communicate, propagate, or transport the program for use by or in connection with the instruction execution system, apparatus, or device.

A data processing system suitable for storing and/or executing program code may include at least one processor coupled directly or indirectly to memory elements through a system bus. The memory elements can include local memory employed during actual execution of the program code, bulk storage, and cache memories that provide temporary storage of at least some program code in order to reduce the number of times code must be retrieved from bulk storage during execution. Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems, storage devices, remote printers, etc., through intervening private and/or public networks. Wireless (e.g., Wi-Fi™) transceivers, Ethernet adapters, and modems, are just a few examples of network adapters. The private and public networks may have any number of configurations and/or topologies. Data may be transmitted between these devices via the networks using a variety of different communication protocols including, for example, various Internet layer, transport layer, or application layer protocols. For example, data may be transmitted via the networks using transmission control protocol/Internet protocol (TCP/IP), user datagram protocol (UDP), transmission control protocol (TCP), hypertext transfer protocol (HTTP), secure hypertext transfer protocol (HTTPS), dynamic adaptive streaming over HTTP (DASH), real-time streaming protocol (RTSP), real-time transport protocol (RTP) and the real-time transport control protocol (RTCP), voice over Internet protocol (VOIP), file transfer protocol (FTP), WebSocket (WS), wireless access protocol (WAP), various messaging protocols (SMS, MMS, XMS, IMAP, SMTP, POP, WebDAV, etc.), or other known protocols.

Finally, the structure, algorithms, and/or interfaces presented herein are not inherently related to any particular computer or other apparatus. Various general-purpose systems may be used with programs in accordance with the teachings herein, or it may prove convenient to construct more specialized apparatus to perform the required method blocks. The required structure for a variety of these systems will appear from the description above. In addition, the specification is not described with reference to any particular programming language. It will be appreciated that a variety of programming languages may be used to implement the teachings of the specification as described herein.

The foregoing description has been presented for the purposes of illustration and description. It is not intended to be exhaustive or to limit the specification to the precise form disclosed. Many modifications and variations are possible in light of the above teaching. It is intended that the scope of the disclosure be limited not by this detailed description, but rather by the claims of this application. As will be understood by those familiar with the art, the specification may be embodied in other specific forms without departing from the spirit or essential characteristics thereof. Likewise, the particular naming and division of the modules, routines, features, attributes, methodologies and other aspects are not mandatory or significant, and the mechanisms that implement the specification or its features may have different names, divisions and/or formats.

Furthermore, the modules, routines, features, attributes, methodologies and other aspects of the disclosure can be implemented as software, hardware, firmware, or any combination of the foregoing. Also, wherever an element, an example of which is a module, of the specification is implemented as software, the element can be implemented as a standalone program, as part of a larger program, as a plurality of separate programs, as a statically or dynamically linked library, as a kernel loadable module, as a device driver, and/or in every and any other way known now or in the future. Additionally, the disclosure is in no way limited to implementation in any specific programming language, or for any specific operating system or environment. Accordingly, the disclosure is intended to be illustrative, but not limiting, of the scope of the subject matter set forth in the following claims.

What is claimed is:

1. A plug-in ventilation control device comprising:
one or more sensors configured to collect data, wherein the data comprises at least one of temperature, light, sound, humidity, communication from exterior devices, and power availability;
a plug configured to receive power from a house mains source power supply; and
a receptacle configured to make electrical connection to venting component;
wherein the control device is configured to cause the ventilation component to receive power from the house mains source responsive to the data.

2. The device of claim 1, wherein the device is configured to cause the venting component to receive power from the power supply in response to the data including environmental data corresponding to an event occurring.

3. The device of claim 2, wherein the event is a positive change in the environmental data shown as an environmental temperature and a humidity beyond a threshold over a short period of time corresponding to a shower running.

4. The device of claim 2, wherein the event is environmental data corresponding to a flushing of a toilet.

5. The device of claim 1, wherein the device is configured to cause the venting component to receive power from the power supply for a determined period of time in response to power availability at the power supply.

6. The device of claim 1, further comprising:
a cover, configured to fit over a space for a ventilation opening, wherein the cover mounts to a surface surrounding the ventilation opening using pins and or nails driven into the surface around the ventilation opening at angles sufficient to keep the cover stationary.

7. The device of claim 3 wherein the device is contained within a cover for a ventilation opening that mounts to a surface surrounding the ventilation opening.

8. The device of claim 4 wherein the device is contained within a cover for a ventilation opening that mounts to a surface surrounding the ventilation opening.

9. The device of claim 5 wherein the device is contained within a cover for a ventilation opening that mounts to a surface surrounding the ventilation opening.

10. The device of claim 1, wherein the device further comprises one or more peripheral components including one or more of a music playback system, a speaker, a microphone, and a light.

11. The device of claim 1, wherein the device further includes a built-in nightlight.

12. The device of claim 6, wherein the device further comprises a built-in dispersal system for aromatic oils and substances.

13. The device of claim 6 wherein the device attaches to the surface surrounding the ventilation opening by means of an adhesive.

14. The device of claim 6 wherein the device attaches to the surface surrounding the ventilation opening by means of screws driven into the surface around the ventilation opening.

15. The device of claim 6 wherein the cover has built-in lighting elements for room illumination.

16. The device of claim 6 wherein the cover has built-in colored lighting elements for room illumination or ambiance.

17. The device of claim 6 wherein the cover has built-in UV lighting for disinfection of room surfaces.

18. The device of claim 6 wherein the cover has various peripheral components built-in including one or more of a music playback system, a speaker, a microphone, and a voice assistant.

19. The device of claim 1, further comprising a remote device placed in one or more of within a toilet tank and upon a toilet, the remote device being configured to transmit a data signal upon the toilet being flushed.

20. A plug-in ventilation control device comprising:
one or more sensors configured to collect sound data;
a plug configured to receive power from a house mains source; and
a receptacle configured to make electrical connection to ventilation component;
wherein the control device is configured to cause the ventilation component to receive power from the house mains source responsive to determining that the collected sound data matches a toilet flush.
